(19) United States
(12) Patent Application Publication
(10) Pub. No.: US 2006/0270023 A1
(43) Pub. Date: Nov. 30, 2006

(54) Three-dimentioal, flexible cell growth substrate and related methods

(75) Inventors: Philip R. LeDuc, Pittsburgh, PA (US); James D. Kubicek, Pittsburgh, PA (US); Stephanie Bretsford, Arlington, MA (US)

Correspondence Address: KIRKPATRICK & LOCKHART NICHOLSON GRAHAM LLP, 535 SMITHFIELD STREET, PITTSBURGH, PA, 15222, US

(73) Assignee: CARNEGE MELLON UNIVERSITY, Pittsburg, PA (US)

(21) Appl. No.: 10/553,249

(22) PCT Filed: Apr. 16, 2004
(86) PCT No.: PCT/US04/11796

§ 371 Date: Aug. 7, 2006

Related U.S. Application Data
(60) Provisional application No. 60/464,010, filed on Apr. 18, 2003.

(51) Int. Cl. C12M 3/00

(52) U.S. Cl. 435/289.1

(57) ABSTRACT

An engineered cell growth substrate is provided along with an apparatus containing the substrate. The substrate contains features, including, without limitation: an extracellular matrix mimetic, areas of differing elasticity moduli and/or 3-dimensional engineered formations, such as grooves or protuberances. Methods of fabricating the cell growth substrate, methods of growing cells on the substrate, and methods of evaluating analytes on cells on the substrate also are provided.

This application claims priority to U.S. Provisional Patent Application No. 60/464,010, filed Apr. 18, 2004, which is incorporated by reference in its entirety.

### BACKGROUND

A flexible, three-dimensional cell growth substrate and methods for culturing cells are provided.

Cells are propagated or otherwise cultured in vitro for a number of reasons, including, without limitation: to characterize the cells, for example in basic scientific research or to diagnose a disease (pathology); to test the sensitivity of the cells to a drug or biologic; to manipulate the cells for a desired purpose, for instance by gene transfer or by cell fusion; or to grow a tissue for transplantation, for instance to produce a skin graft.

Traditional methods for culturing cells include placing the cells in a dish, plate, flask or roller bottle in a suitable growth medium. Cells traditionally are placed into a dish, plate, flask or roller bottle in medium and are maintained, for example, in an incubator at a specified temperature, humidity and CO₂ level. Plated cells typically adhere to the plate and, to dissociate the cells from the plate for propagation, the cells often are treated with a protease, such as trypsin. Some cells, such as embryonic stem cells are cultured on a layer of fibroblasts or other cells. Alternately, cells may be suspended in media in spinner bottles and are propagated by removing aliquots from the cell culture and placing the cells in other spinner bottles.

As the science of cell propagation has advanced, it had been found that mechanical stress, when applied to cells, could aid in the propagation of cells, including cells that are not capable of propagation in vitro and in better simulating in vivo growth conditions. U.S. Pat. No. 4,822,741 describes a flexible, derivatized polyorganosiloxane membrane upon which cells can be grown. The membrane is affixed peripherally to a rigid support and is flexed rhythmically by application of a vacuum to the side of the membrane opposite that of the cells.

The link between mechanics and biochemistry in cellular and molecular research has been studied to provide insights into many areas such as mechanotransduction, polymer physics, and bioengineering (LeDuc P, Haber C, Bao G, Wirtz D, Dynamics of individual flexible polymers in a shear flow. Nature 1999; 399: 564-566 and Wang N, Butler J P, Ingber D E, Mechanotransduction across the cell surface and through the cytoskeleton. Science 1993; 260: 1124-1127). Intensive studies of the link between the mechanics and biochemistry of cells also have provided insights into physiological mechanotransduction, including gravity sensation, audio-sensory channels, and baro-reception, and has stimulated research in related areas, including, without limitation, polymer physics and bioengineering. For example in mechanotransduction, the application of shear stress to cells has assisted in the contributed to an understanding of the regulation of canonical intracellular signal-transduction pathways, for example and without limitation, mitogen-activated protein kinase pathways such as p38 and jun-n-terminal kinase (Ferrer I, Blanco R, Carmona M, Puig B, Barrachina M, Gomez C, Ambrosio S. Active, phosphorylation-dependent mitogen-activated protein kinase (MAPK/ERK), stress-activated protein kinase/c-Jun N-terminal kinase (SAPK/JNK), and p38 kinase expression in Parkinson's disease and Dementia with Lewy bodies. J Neural Transm 2001; 108: 1383-1396; Li C, HuY, Mayr M, Xu Q, Cyclic strain stress-induced mitogen-activated protein kinase (MAPK) phosphatase 1 expression in vascular smooth muscle cells is regulated by Ras/Rac-MAPK pathways. J Biol Chem 1999; 274: 25273-25280 and Shrode L D, Rubie E A, Woodgett J R, Grinstein S, Cytosolic alkalinization increases stress-activated protein kinase/c-Jun NH2-terminal kinase (SAPK/JNK) activity and p38 mitogen-activated protein kinase activity by a calcium-independent mechanism J Biol Chem 1997; 272: 13653-13659) as well as the genome through alterations in gene expression profiles (Garcia-Cardena G, Comander J L Blackman B R, Anderson K R, Gimbrone M A, Mechanosensitive endothelial gene expression profiles: scripts for the role of hemodynamics in atherogenesis? Ann N Y Acad Sci 2001; 947: 1-6; Topper J N, Gimbrone M A, Jr., Blood flow and vascular gene expression: fluid shear stress as a modulator of endothelial phenotype. Mol Med Today 1999; 5: 40-46 and Resnick N, Yahav H, Khachigian L M, Collins T, Anderson K R, Dewey F C, Gimbrone M A, Jr., Endothelial gene regulation by laminar shear stress. Adv Exp Med Biol 1997; 430: 155-164) and the regulation of the second messenger, cyclic adenosine monophosphate (Meyer, C. J., Alenghat, F. J., Rim, P., Fong, J. H., Fabry, B. & Ingber, D. E. (2000) Nat Cell Biol 2, 666-8; Garcia-Cardena, G., Comander, J. I., Blackman, B. R., Anderson, K. R. & Gimbrone, M. A. (2001) Ann N Y Acad Sci 947, 1-6; Topper, J. N. & Gimbrone, M. A., Jr. (1999) Mol Med Today 5, 40-6; and Resnick, N., Yahav, H., Khachigian, L. M., Collins, T., Anderson, K. R., Dewey, F. C. & Gimbrone, M. A., Jr. (1997) Adv Exp Med Biol 430, 155-64). Further, ultimate mammalian cell behavior is linked to mechanical stimuli, since the application of stress influences proliferation, differentiation and apoptosis (Matsuda, N., Morita, N., Matsuda, K. & Watanabe, M. (1998) Biochem Biophys Res Commun 249,350-4; Liu, S. Q., Ruan, Y. Y., Tang, D., Li, Y. C., Goldman, J. & Zhong, L. (2002) Biomech Model Mechanobiol 1, 17-27; Weyts, F. A., Bosmans, B., Niesing, R., Leeuwen, J. P. & Weinans, H. (2003) Calcif Tissue Int 72, 505-12; Hammerschmidt, S., Kuhn, H., Grasenack, T., Gessner, C. & Wirtz, H. (2003) Am J Respir Cell Mol Biol.; Husse, B., Sopart, A. & Isenberg, G. (2003) Am J Physiol Heart Circ Physiol 285, Hi521-7; and Chess, P. R., Toia, L. & Finkelstein, J. N. (2000) Am J Physiol Lung Cell Mol Physiol 279, L43-51). Prior observations have largely been made in in vitro experiments using limited environmental constraints such as prescribed material scaffolding or two-dimensional mechanical-stimulation materials (Levenberg, S., Huang, N. F., Lavik, E., Rogers, A. B., Itskovitz-Eldor, J. & Langer, R (2003) Proc Natl Acad Sci USA 100, 12741-6; Sumpio, B. E., Banes, A. J., Levin, L. G. & Johnson, G., Jr. (1987) J Vasc Surg 6, 252-6; Camargo, M. J., Sumpio, B. E. & Maack, T. (1984) Am J Physiol 247, F656-64; and Boitano, S., Sanderson, M. J. & Dirksen, E. R (1994) J Cell Sci 107 (Pt 11), 3037-44).

New technologies can be utilized in medical stimulation that help provide new insights into the response of single cells and cell populations. For example, micropipette aspirations devices have improved the understanding of membrane mechanics including the interconnection between the membrane of the outer hair cell with its cortical lattice, the cell cycle regulated viscoelastic properties for hepatocellular carcinoma cells, and the volume differential behavior of red blood cells under an anisotonic environment (Morimoto N, Raphael R M, Nygren A, Brownell W E, Excess plasma membrane and effects of ionic amphipaths on mechanics of outer hair cell lateral wall. Am J Physiol Cell Physiol 2002; 282: C1076-1086; Engstrom K G, Meiselman H J, Combined use of micropipette aspiration and perifusion for studying red blood cell volume regulation. Cytometry 1997; 27: 345-352 and Wang J H, Goldschmidt-Clermont P, Wifle J, Yin F C, Specificity of endothelial cell reorientation in response to cyclic mechanical stretching. J Biomech 2001; 34: 1563-1572). Further, fluid shearing devices have helped uncover protein-specific attachment sites and the contributions of the cytoskeleton (Dekker R J, van Soest S, Fontijn R D, Salamanca S, de Groot P G, VanBavel E, Pannekoek H, Horrevoets A J, Prolonged fluid shear stress induces a distinct set of endothelial cell genes, most specifically lung Kruppel-like factor (KLF2). Blood 2002; 100: 1689-1698; Malek A M, Izumo S, Mechanism of endothelial cell shape change and cytoskeletal remodeling in response to fluid shear stress. J Cell Sci 1996; 109 (Pt 4): 713-726; Schnittler H J, Schneider S W, Raifer H, Luo F. Dieterich P, Just L Aktories K Role of actin filaments in endothelial cell-cell adhesion and membrane stability under fluid shear stress. Pflugers Arch 2001; 442: 675-687; Kano Y, Katoh K, Fujiwara K, Lateral zone of cell-cell adhesion as the major fluid shear stress-related signal transduction site. Circ Res 2000; 86: 425433; Truskey G A, Pirone J S, The effect of fluid shear stress upon cell adhesion to fibronectin-treated surfaces. J Biomed Mater Res 1990; 24: 1333-1353 and van Kooten T G, Schakenraad J M, van der Mei H C, Dekker A, Kirkpatrick C J, Busscher H J, Fluid shear induced endothelial cell detachment from glass-influence of adhesion time and shear stress. Med Eng Phys 1994; 16: 506-512). In current experimental devices for mechanical stimulation by cell stretching, planar two-dimensional deformations are imposed. However, in vivo cells are observed to experience three-dimensional constraints including endothelial cells in blood vessels and fibroblasts in regions of local mechanical deformation. Currently there is a lack of devices for accurately producing these complex three-dimensional mechanical perturbations.

### SUMMARY OF THE INVENTION

A cell growth substrates and apparatus containing such substrates are therefore provided. In one embodiment, a cell growth apparatus is provided comprising a cell growth chamber having an interior side and an exterior side and comprising a wall and a base defining an interior volume, the cell growth chamber comprising an elastomeric growth substrate comprising an elastomeric membrane of a first material that comprises a first portion having a first elasticity and a second portion having a second elasticity.

An elastomeric cell growth substrate also is provided comprising comprising an elastomeric membrane of a first material that comprises a first portion having a first elasticity and a second portion having a second elasticity.

A cell growth apparatus is provided comprising a cell growth chamber having an interior side and an exterior side and comprising a wall and a base defining an interior volume, the cell growth chamber comprising an elastomeric growth substrate comprising an elastomeric membrane of a first material having an interior side and an exterior side, wherein the elastomeric membrane is at least partially coated with an extracellular matrix-mimetic.

A cell growth substrate also is provided, comprising an elastomeric membrane of a first material that is at least partially coated with an extracellular matrix-mimetic.

A method of producing an elastomeric cell growth substrate is provided, comprising coating at least a portion of an elastomeric membrane with and extracellular matrix mimetic.

A second method of producing an elastomeric cell growth substrate is provided, comprising preparing an elastomeric membrane of a first material that comprises a first portion having a first elasticity and a second portion having a second elasticity.

A method of culturing cells is provided, comprising growing cells in a suitable cell growth medium in a cell growth apparatus comprising a cell growth chamber having an interior side and an exterior side and comprising a wall and a base defining an interior volume, the cell growth chamber comprising an elastomeric growth substrate comprising an elastomeric membrane of a first material that comprises a first portion having a first elasticity and a second portion having a second elasticity; and flexing the substrate while the cells are growing.

Another method of culturing cells is provided, comprising growing cells in a suitable medium in a cell growth apparatus comprising a cell growth chamber having an interior side and an exterior side and comprising a wall and a base defining an interior volume, the cell growth chamber comprising an elastomeric growth substrate comprising an elastomeric membrane of a first material that is at least partially coated with an extracellular matrix-mimetic; and flexing the substrate while the cells are growing.

Still yet another method of culturing cells is provided, comprising growing cells in a suitable cell growth medium in a cell growth apparatus comprising a cell growth chamber having an interior side and an exterior side and comprising a wall and a base defining an interior volume, the cell growth chamber comprising an elastomeric growth substrate comprising a first elastomeric membrane and a removable second elastomeric membrane having one or more protuberances contacting the first elastomeric membrane or openings, the periphery of which contact the first elastomeric membrane; flexing the substrate while the cells are growing; and removing the second elastomeric membrane.

### BRIEF DESCRIEPTION OF THE DRAWINGS

FIGS. 1A through 1G depict one embodiment of the cell growth apparatus described herein in which positive pressure is used to flex the elastomeric substrate.

FIGS. 2A and 2B depict a second embodiment of the cell growth apparatus described herein in which negative pressure (vacuum) is used to flex the elastomeric substrate.

FIGS. 3A through 3E depict a variant embodiment of the cell growth apparatus shown in FIGS. 1A-1G in which different masks are used to define the shape of the cell growth membrane and to constrain the stretching profile of the membrane.

FIG. 4A is a photomicrograph of cells growing on one embodiment of the elastomeric substrate described herein that is coated with an extracellular matrix mimetic.

FIG. 4B is a schematic diagram showing the pattern of deposition of fibronectin on the membranes shown in the photomicrograph of FIG. 4C. The scale bar in FIG. 4C is 30 μm.

FIG. 5A is a graph of the theoretical results of the flexible membrane strain versus pressure. The vertical displacement of the center point on the flexible membrane was determined through analytical calculation with an exact solution of 26 terms (solid triangle), a triangular assumption (open triangles, a bulge test solution (open squares) and the Ramanujan's ellipse approximation (solid diamonds).

FIG. 5B is a graph of the experimental results of interferometric measurements of deflection versus pressure. The vertical displacement of the center point on the flexible membrane was measured as a function of pressure.

FIG. 6A is a photomicrograph of an engineered elastomeric membrane having channels on its surface. The scale bar is 500 μm.

FIG. 6B is a schematic diagram of a layered engineered structure having internal channels.

FIG. 6C is a photomicrograph of cells grown in channels substantially as shown in FIG. 6A. The scale bar is 30μm.

FIG. 7 is a grayscale version of a color eipfluorescent image of immunolabeled NIH 3T3 fibroblasts on the described membrane. The nucleus is stained blue in the original and the actin Cytoskeleton is stained green in the original with Dapi and fluorescein isothiocyanate phalloidin, respectively. Scale bar=15 μm.

FIG. 8 shows a metallic grid embedded in polydimethylsiloxane (PDMS). This μm-scale grid creates gradients of deformation of membranes for cell stretching (inset). The scale bar is 50 μm.

FIG. 9 is a graph showing interferometric measurements of vertical deflection for pressures from 0 to 0.4 lb/in² with a PDMS membrane 1 mm in thickness. The deflection is measured at the center of the circular membrane as shown in the inset. The error bars represent the standard deviation.

FIGS. 10A, 10B, 11A and 11B shows experimental and mathematical-simulation results of displacement profiles under uniform pressure. FIG. 10A shows MicroVal coordinate measurements of vertical deflection with respect to horizontal position across the membrane surface under pressure with a PDMS membrane 1 mm in thickness. FIG. 10B shows displacements of the membrane, modeled using the finite-element method with shell elements in ANSYS, of the deflection of homogeneous PDMS under uniform pressure constrained at the periphery of the membrane. The results are differentially shaded for the displacements, with the greatest displacements at the center of the membrane. FIG. 11A shows membrane displacement due to the gradient of elastic moduli in the device. MicroVal coordinate measurements of vertical deflection with respect to horizontal position across the surface of a membrane with a rectangular nylon section embedded in the membrane at 6.8 mm on the graph. FIG. 11B shows a side view of a membrane showing the displacements modeled as in FIG. 10B with an embedded rectangular section.

### DETAILED DESCRIPTION OF THE PREFERRED EMBODIMENTS

As discussed above, prior observations on cell behavior have largely been made in in vitro experiments using limited environmental constraints such as prescribed material scaffolding or two-dimensional mechanical-stimulation materials. These methods, however, were unable to simulate the complex multidimensional array of environmental stimulation, including mechanical, chemical, and scaffolding, which influence cellular responses in physiological systems. This lack of effective methods to explore cell behavior in an accurate three-dimensional environment has hampered progress in our understanding of the normal cellular response to mechanical forces in living tissue.

Mechanical stimulation of cells has been shown to affect cellular behavior from the molecular scale to ultimate cell fate including apoptosis and proliferation. The complex spatiotemporal organization of soluble and solid-state cellular and molecular interactions dictates the physiological function of cells. These resultant localized behaviors are manifestations of an integrated response from a multi-variable three-dimensional cellular environment.

Mechanical stimulation is mainly transmitted through a family of transmembrane proteins, the integrins, which are linked intracellularly to a series of proteins including talin, vinculin, and paxillin. These proteins form focal adhesion complexes around the integrins upon their engagement by extracellular ligands in the extracellular matrix (ECM), and connect to actin filaments. Focal adhesion complexes, therefore, represent a junction for force transmission from the ECM to the intracellular cytoskeleton. This mechanism for force transfer from the extracellular arena to the intracellular molecular interactions allows signal communication to have multi-variable constraints. However, the integration of this mechanical connection with the scaffolding system of the cell attachment along with the biochemical environment which is inherent in mammalian cell systems requires the simultaneous control of all components for realistic results to be extracted.

To address this multi-variable control dilemma, a pressure-driven cell-stretching device has been developed. This device constrains an elastomeric membrane to modulate the mechanical stimulation of cells through their ECM connections while also integrating scaffolding and chemical control to reproduce a physiologically functional environment The interconnected mechanical, chemical, and structural stimuli are examined by controlling the cellular milieu through mechanical deformation, spatial confinement of attachments, and control of the cell-extracellular matrix connections. The integration of fabricated structures and molecular patterning allows the introduction of space and time dependent parameters by combining mechanical stimulation, biochemical regulation, and scaffolding design. The method is applied to stimulate single cells and cell populations to examine the cellular interactions with spatiotemporal control. This research provides the capacity to elucidate the emergence of biological pattern and tissue formation.

As used herein, the term “extracellular matrix mimetic” refers to a composition that mimics an in vivo extracellular matrix. Examples of extracellular matrix mimetics include, without limitation, fibronectin, vitronectin, collagen, laminin, polyoactide) ALA), poly(lactide-co-glycolide)(PLGA), Matrigel (BD Biosciences) and PuraMatrix self-assembling polypeptide scaffolding, commercially available from 3DM, Inc. of Cambridge, Mass. and as disclosed in U.S. Pat. Nos. 5,670,483, 5,955,343 and 6,368,877. Natural extracellular matrix compositions are considered to be within the definition of extracellular matrix mimetics. Extracellular matrix mimetics are members of a broader class of compositions that are referred to herein as “adhesion promoters,” that further include as a class, without limitation: anti-surface protein antibodies, including anti-alpha integrin, anti-beta integrin, anti-selectin, and anti-cadherin antibodies, and polylysine.

As used herein, the term “adhesion inhibitor” refers to an agent that prevents adhesion of the extracellular matrix mimetic and/or cells to the elastomeric substrate. Non-limiting examples of adhesion inhibitors include bovine serum albumin (BSA) and Pluronic® block compolymers. Pluronic® block copolymers are poly(ethylene oxide)/poly(propylene oxide)/poly(ethylene oxide) triblock polymers. In one embodiment, the triblock polymer comprises at least about 30 propylene oxide residues.

As used herein, the terms “elastic,” “elasticity,” “elastomeric,” and like terms refer to a physical property of the elastomeric substrate, namely the deformability of the elastomeric substrate under mechanical force and the ability of the substrate to retain its original shape when the deforming force is removed. In the context of the cell growth devices described herein, the elastomeric substrate typically is stretched by application of pressure by a fluid (gas or liquid) on the underside, or exterior side, of the substrate, resulting in stretching of the substrate in one or more axes. An elastomeric substrate will deform to a desirable degree, as indicated herein, when force is applied to the substrate.

As used herein, the term “elastic modulus” refers to Young's Modulus and is a measure of the ratio of (a) the uniaxial stress along an axis of the material to (b) the accompanying normal strain along that axis.

As used herein, the term “biodegradable” refers to the ability of a polymer to erode or biodegrade in vivo over time.

As used herein, the term “engineered structural formation” refers to any structure produced within or attached to the elastomeric substrate. The engineered structural formation can be molded into the elastomeric substrate, attached to the substrate or result from the attachment of one or more additional elastomeric layers on the substrate. The engineered structural formation can be, without limitation: an indentation, such as a groove, pit or channel; a passageway, a pocket; and a protuberance, such as a ridge. Any engineered structural formation within the substrate, such as a passageway or pocket can open to a surface of the substrate, typically to a surface of the membrane that opens to the interior of a cell growth chamber comprising the substrate.

As used herein, the term “cell” refers to any cell, more typically eukaryotic cells and most typically mammalian cells. For many uses for the substrates and apparatus described herein, such as for tissue engineering or analyte testing, cells of human origin are preferred. Use of human embryonic stem cells may be preferred for some uses, particularly where tissue differentiation is desired. For other uses, such as for testing of analytes such as drugs, cells of a particular tissue origin may be desired, including cells from tumor biopsies or resections.

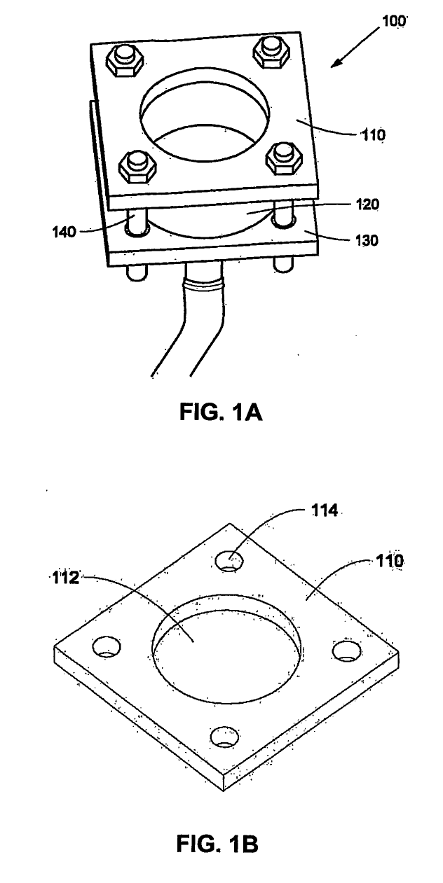

Certain non-limiting embodiments of the present invention are depicted in the accompanying Figures. FIG. 1A, shows one assembled embodiment of device 100. Device 100 includes a pressure plate 110, an annular wall 120 and a base plate 130, which are held in place by bolts 140. Bolts 140 may be substituted with any suitable clamping device or other fastener system that can hold pressure plate 110, an annular wall 120 and a base plate 130 in place and apply pressure between pressure plate 110 and base plate 130. FIG. 1B shows pressure plate 110, including opening 112 and bolt holes 114. FIG. 1C shows annular wall 120. FIGS. 1D and 1E show base plate 130, which includes recess 132, bolt holes 134, opening 136, fitting 138 and peripheral ridge 139. Fitting 138 is configured to receive or otherwise fluidly connect with a pipe or tube. FIG. 1F is a cross section of assembled device 100 as shown in FIG. 1A (bolts 140 not shown), including pressure plate 110, annular wall 120 and base plate 130, including fitting 138 and peripheral ridge 139. Also shown is elastomeric substrate 160 that is held in place by the compression or pressure produced by action of bolts 140 or other clamping system, pinching elastomeric substrate 160 between annular wall 120 and base plate 130. Elastomeric substrate 160 is planar and circular in this embodiment. Annular wall 120 and elastomeric substrate define an interior volume 180 of device 100. A watertight seal is created between elastomeric substrate 160 and annular wall 120 to prevent cell growth media from leaking from interior volume 180. Likewise a fluid-tight (air-tight or liquid-tight) seal is created between elastomeric substrate 160 and base plate 130 to prevent fluid leakage when fluid is forced through opening 136 to flex elastomeric substrate 160. A tube 170 also is shown, through which fluid 190 is introduced into device 100 to flex elastomeric substrate 160.

In use, cell medium and cells are placed in interior volume 180, and the cells are permitted to attach to elastomeric substrate 160. Once cells are attached to elastomeric substrate 160, fluid 190 is forced through opening 136 to flex elastomeric substrate 160, as shown in FIG. 1G. Elastomeric substrate 160 may be flexed in a periodic manner, randomly or in any other temporal pattern. Fluid 190 can be forced through opening 136 by a pump (not shown) thereby applying pressure to and deforming elastomeric substrate 160. The pressure and timing of the application of force to elastomeric substrate 160 can be controlled by any of the many methods known in the art. In one example, solenoid valves control flow of the fluid through tube 170. The solenoid valves can be controlled electronically, for example, by a computer or other equivalent device. One non-limiting method for controlling fluid flow through opening is shown in U.S. Pat. No. 4,822,741. Although that device is used to produce a vacuum in connection with a vacuum source, the device is equally suited to control the application of pressure to device 100.

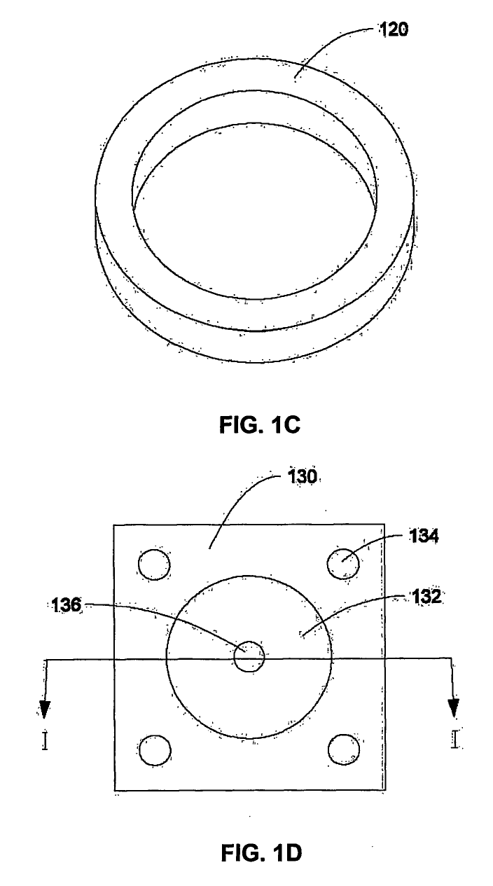

FIGS. 2A and 2B show a second embodiment of the cell growth apparatus. Device 200 includes pressure plate 210, annular wall 220, base plate 230, elastomeric membrane 260 and tube 270. In use a vacuum 290 is applied to device 200, thereby flexing elastomeric 20 membrane 260 toward base plate 230.

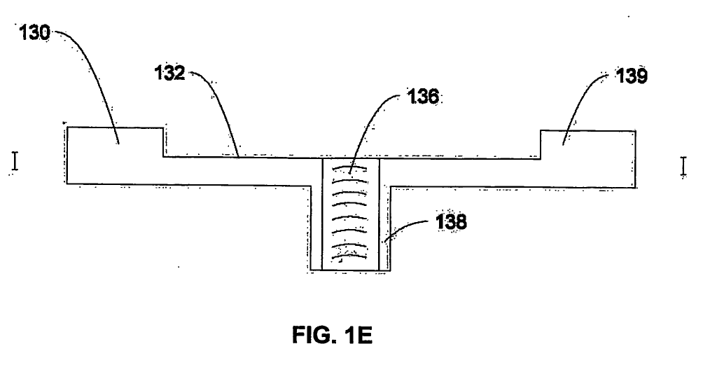

In reference to FIGS. 3A-3E, annular wall 130, shown in FIG. 1C, can be replaced by masks having openings, of different geometries. FIG. 3A shows a mask 320 having an oblong opening 322 having a 2:1 length:width ratio and a ridge 324 adjacent to the periphery of opening 322. FIGS. 3B and 3C show the mask 320 of FIG. 3A assembled into a device 300, configured essentially as the device shown in FIG. 1F, including pressure plate 310, base plate 330, elastomeric membrane 360 and tube 370. Ridge 324 is configured to apply additional anchoring pressure to the periphery of the elastomeric membrane 360. In use, as shown in FIG. 3C, when a fluid is forced into device 300, the membrane is flexed away from base plate 330 with the membrane 360 constrained by ridge 324 of mask 320. FIG. 3D shows a mask 420 having an oblong opening 422 having a 4:1 length:width ratio and a ridge 424 adjacent to the periphery of opening 422. FIG. 3E shows a mask 520 having an oblong opening 522 having a 8:1 length:width ratio and a ridge 524 adjacent to the periphery of opening 522. The masks of FIGS. 3D and 3E can be used in device 300 of FIG. 3C in the same manner as the mask of FIG. 3A.

FIGS. 1-3 depict only a few of an infinite variety of possible physical configurations of the device. A person of skill in the art would be able to modify devices 100, 200, 300 substituting any practical shape for the annular shape of wall 120 and disc shape of membrane 110 as is depicted in FIGS. 1A-1G. In reference to FIGS. 1A-1G, membrane 110 and wall 120 can have other geometries, for example and without limitation, ellipses and squares, and arbitrary shapes so that the stresses imposed on the cell(s) attached to the membrane can vary in one or more axes across the length, breadth, and height of the membrane. Alternately, masks different from those of FIGS. 1C, 3A, 3D and 3E can be used. Either gas or liquid (collectively “fluids”) can be used to cause flexion of membrane 110 in either a positive direction, away from the gas or fluid, by placing positive pressure on membrane 110 or in a negative direction, towards the gas or fluid, by placing a negative pressure on membrane 110, such as by application of a vacuum. Device 110 can have a variety of configurations, with one cell growth chamber or multiple chambers. U.S. Pat. Nos. 6,057,150 and 6,472,202, disclose examples of different devices useful applying positive or negative pressure on the membrane. The elastomeric substrate optionally can be glued to wall 120 and/or base 130, according to known methods and using known adhesives, sealants or lamination techniques, potentially removing the need for pressure plate 140 and the need to apply a sealing compression to hold and seal the substrate in place.

In one embodiment of the device depicted in FIGS. 1-3, a polyorganosiloxane membrane, such as, for example and without limitation, a polydimethylsiloxane (PDMS) membrane, coated with an extracellular matrix mimetic is utilized to mechanically stimulate cells by virtue of its quasi-elastic stress-strain behavior. A regulator can apply pressure ranging from, for example and without limitation, −15 to +15 lb/in² to the bottom surface of the membrane to modulate the deformation induced. Cells typically are initially cultured on the membrane to form an adherent monolayer and are then placed in the device. Alternately, the cells can be cultured in the intact device prior to application of pressure to the membrane. A controlled increase in the pressure on the lower surface of a radially symmetric membrane in the device creates an associated equibiaxial stretch due to the fixed-displacement boundary conditions. The strain is transferred to cells through their basal side attachments where the extracellular matrix mimetic-coated PDMS surface allows attachment and spreading of single cells. Since the differential pressure is physically separated from the surface of the membrane-cell attachment, an associated deformation of the flexible membrane strains the cells mechanically. This approach can be used with various porous and elastic materials, including biodegradable membranes, for engineered tissues and three-dimensional scaffolding control (see, e.g., Sansson, K., Haegerstrand, A. & Kratz, G. (2001) Scand J Plast Reconstr Surg Hand Surg 35,369-75; Wang, J. H., Yao, C. H., Chuang, W. Y. & Young, T. H. (2000) J Biomed Mater Res 51, 761-70; and Wang, Y., Ameer, G. A., Sheppard, B. J. & Langer, R. (2002) Nat Biotechnol 20, 602-6). Although the substrate that is being stretched by pressurization is planar, embedded fabricated networks and composite organization within the membrane of varied elasticity produce a three-dimensional strain on the cells in the device. For example, cells cultured in fabricated structures inside the membrane can experience stress at their basal and apical surfaces simultaneously.

The elastomeric membrane can be a single layer or can have multiple layers, each layer, independently having the same or a different 3-dimensional structure and composition. In one embodiment, a solid base membrane is prepared from polydimethylsiloxane. Alternative polymers that can be used in place of polydimethylsiloxane, such as, without limitation, other siloxane products, elastomeric silicone rubbers and hydrogels, as are known in the art. Erodeable elastomeric polymers, such as, without limitation, a poly(glycerol-sebacate) polymer, such as a polymer described in Wang Y, Ameer G, Sheppard B, Langer R, A Tough Biodegradable Elastomer, Nature Biotechnology 2002; 20:602-606, also may be used.

In a single-layer substrate, a variety of parameters may be modified to create a suitable cell growth substrate. One parameter is to attach an extracellular matrix mimetic to the surface of the elastomeric membrane. The extracellular matrix mimetic may coat the entire surface of the membrane or only a portion thereof. The extracellular matrix mimetic may be patterned on the surface of the membrane to achieve a desired cell growth pattern. Other adhesion promoters also may be patterned onto the membrane in addition to, or as a substitute for the extracellular matrix mimetic. Cell adhesion can be blocked from areas of the membrane by applying an adhesion inhibitor, such as, without limitation, BSA and Pluronic® polymers. The extracellular matrix mimetics, adhesion promoters and adhesion inhibitors also can be patterned by microfluidic methods, as are well known in the art, to coat all or part of any engineered structure of the substrate. For example, and without limitation, a passageway in the substrate or a groove on the surface of the substrate can be coated selectively with an extracellular matrix mimetic or an adhesion promoter, to promote cell growth within the passageway or groove.

The thickness of the membrane also can be varied over the membrane, with a portion (area) of the membrane being thicker than another portion, thereby producing two or more portions of the membrane having differing elasticity. Thickness can be modified by curing the membrane in a mold having the desired profile or other methods as are known in the fabrication arts.

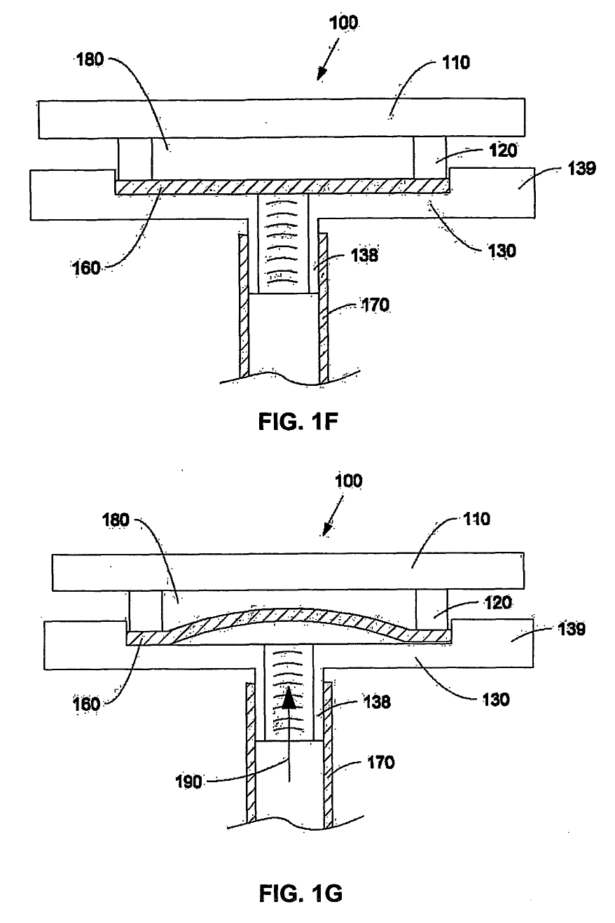

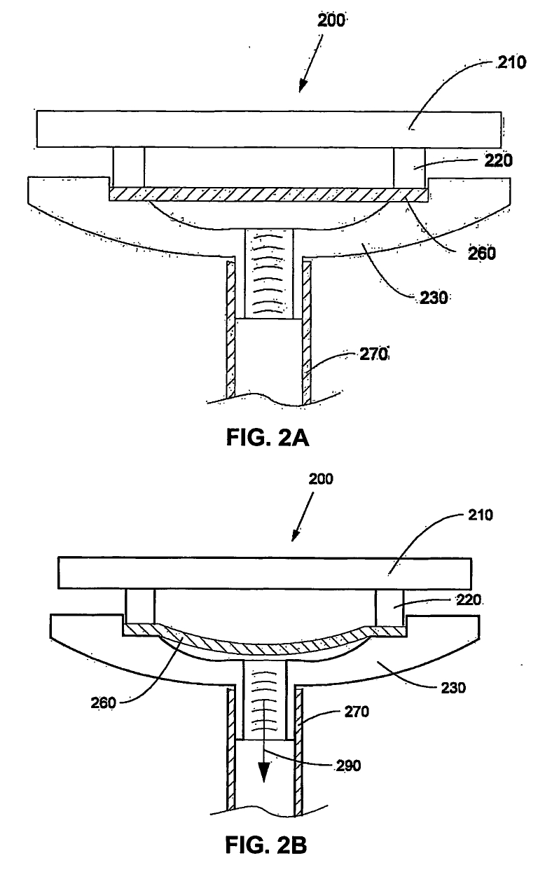

Engineered structures can be introduced in the membrane by creating a suitable mold having the desired profile. One method for producing a highly detailed mold, and for preparing PDMS membranes having precisely defined microstructure is descried in LeDuc et al., Use of Micropattemed Adhesive Surfaces for Control of Cell Behavior, Methods in Cell Biology, 69:395-401. Briefly, in one embodiment, the design for the micropattern is drawn to scale using a commercial computer drawing package and the micropattern is used to fabricate a photomask according to conventional methods. A silicon wafer is coated with a photoresist layer, the photomask is overlaid on the wafer and the wafer is exposed to ultraviolet (UV) light. The photoresist is then chemically developed, resulting in dissolution of UV-exposed regions of the photoresist layer. The resulting bas-relief pattern is then exposed, for example and without limitation, to vapors of (tridecafluoro-1,1,2,2,-tetrahydrooctyl)-1-trichlorosilane to reduce its adhesivity to PDMS. Uncured PDMS is then poured over the pattern and is cured at 60° C. for two hours. To achieve uniform membrane thickness, the silicon wafer can be spun in a Spin Coater at 500-8000 rpm. Once cured, the PDMS is removed from the silicon wafer and is sterilized with ethanol in anticipation of its use as a cell growth substrate. All or part of the surface of the membrane can be coated with an extracellular matrix mimetic, for example as is shown in FIGS. 4A-4C, described below. Variations in and modifications to this lithographic process for preparing engineered structures in the membrane would be apparent to those of skill in the art and are considered to be within the scope of the present invention.

Other materials having a different elastic modulus than the elastomeric membrane may be embedded within or affixed to the elastomeric membrane. For example and without limitation, a nylon mesh, a stainless steel mesh, a copper mesh, a ceramic mesh or a non-woven material may be placed on uncured PDMS or PDMS can be poured over the mesh in a suitable mold to embed the mesh within the membrane. Alternately, the mesh can be laminated between two elastomeric layers. The material having a different elastic modulus can literally take any form or shape and can comprise virtually any biocompatible material, including without limitation: meshes, sheets, discs, gauzes and wires.

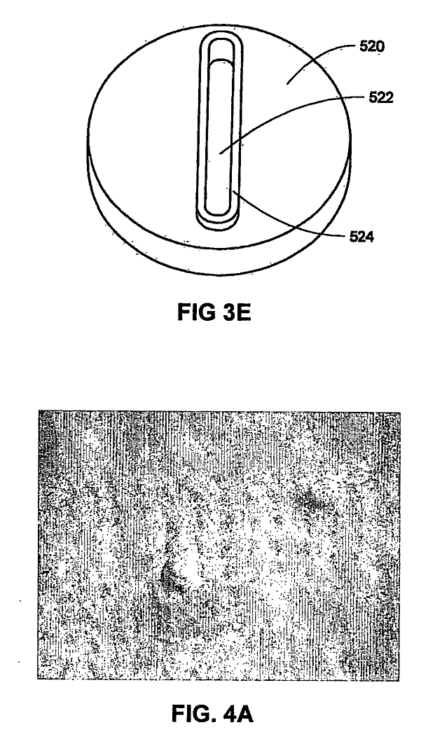

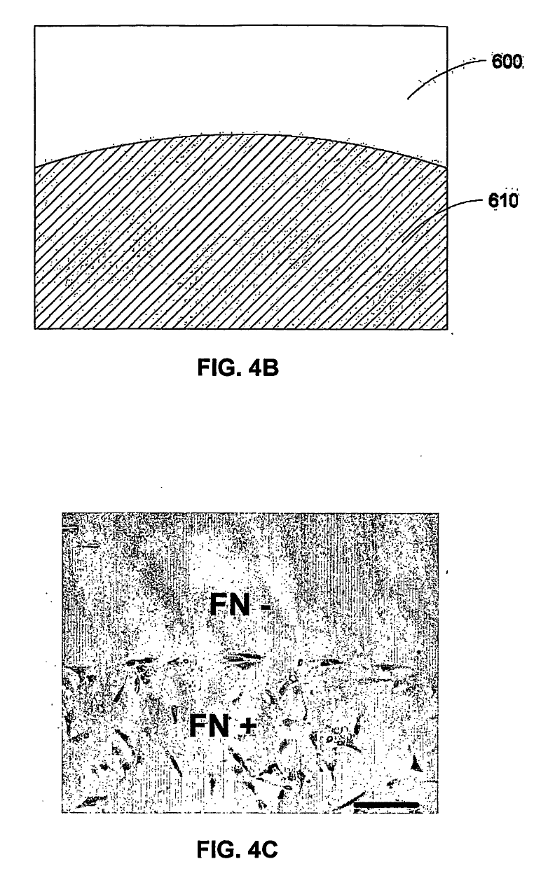

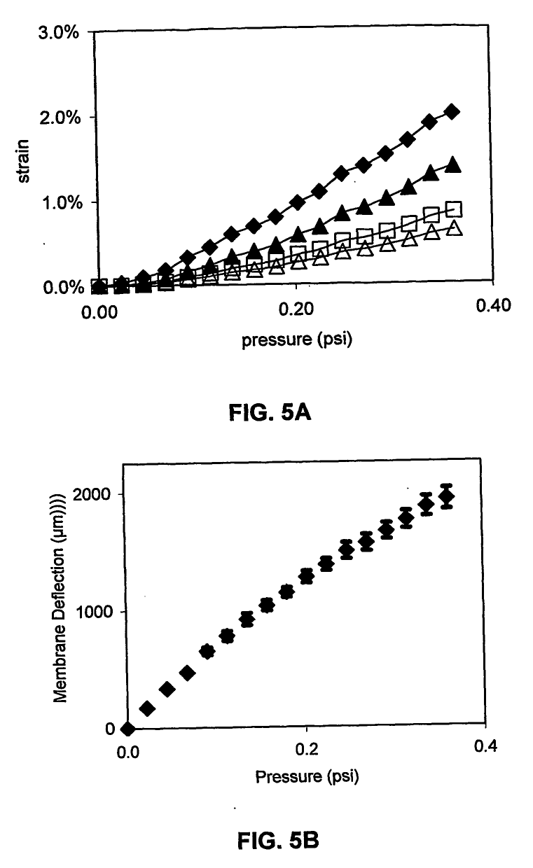

Multi-layered substrates can be prepared by attaching or laminating two or more elastomeric layers. The layers can be of the same or different material with the same or different elastic moduli and can be the same or different sizes. Variations in elastic modulus on a layer-by-layer basis, or over the entire membrane, can result in differential mechanical strain on the cells at different points within or upon the membrane. Passageways can be formed within an elastomeric substrate by preparing a first membrane with grooves engineered on its surface and overlaying a second layer with a planar surface atop the grooved surface, substantially as shown in FIG. 6B, discussed in further detail below. As mentioned above, a mesh can be laminated at points between two layers to produce a local area having a different elastic modulus than the rest of the substrate. Any engineered structure in the 3-dimensional matrix formed by these methods can be coated selectively with an extracellular matrix mimetic or an adhesion inhibitor to dictate where cell growth is or is not to occur. As is evident, multiple layers can be produced with different physical structures and degrees of deformability. Layers may be laminated together or otherwise attached by heat curing, by the application of an adhesive or by any method known in the art.

In one further embodiment, two membranes are placed into conformal contact, for example in device 100 shown in FIGS. 1A-1G with regard to elastomeric membrane 160. A first membrane can include protuberances on a side facing a second membrane such that the protuberances of the first membrane contact the other membrane to block local cell growth on that other membrane. Once cells are grown to a sufficient density, the first layer is removed, leaving a patterned cell population. The protuberances in the first membrane can be prepared by the lithographic methods described above. The first and/or second membrane can include channels that permit passage of cells and cell growth medium into the space between the two membranes. In a variation of this embodiment, first membrane can include perforations, rather than protuberances, to guide deposit and growth of the cells.

Cells, extracellular matrix mimetics, adhesion promoters and/or adhesion inhibitors can be patterned using the above-described engineered membrane having one or more protuberances. A first elastomeric membrane can be overlaid with a constraining layer having one or more protuberances so that the one or more protuberances of the engineered membrane contact the surface of the first elastomeric membrane. When the constraining layer is used to train cell growth, it typically is coated with an adhesion inhibitor and/or is fabricated of a material to which cells do not adhere. Cells, extracellular matrix mimetics, adhesion promoters and/or adhesion inhibitors can then be applied to the structure, thereby coating surfaces and/or engineered structures of the first elastomeric membrane. Once the cells, extracellular matrix mimetics, adhesion promoters and/or adhesion inhibitors have had sufficient time to absorb, adsorb and/or otherwise attach to the surface of the first elastomeric membrane, the constraining layer can be removed. Multiple constraining layers may be used to pattern cells, extracellular matrix mimetics, adhesion promoters and/or adhesion inhibitors on a membrane. The constraining layers may be stacked, revealing progressively larger areas upon removal of each constraining layer, and/or the constraning layer cover different portions of the first membrane.

In another embodiment, the constraining layer also can be in the form of a mask, having openings through which cells, extracellular matrix mimetics, adhesion promoters and/or adhesion inhibitors can pass to adhere onto the first elastomeric membrane. As above, once the cells, extracellular matrix mimetics, adhesion promoters and/or adhesion inhibitors are sufficiently affixed to the first membrane the constraining layer is removed, permitting application of different cells, extracellular matrix mimetics, adhesion promoters and/or adhesion inhibitors, or even use of a different constraining layer.

In one additional embodiment, an analyte is tested for its effect on cells grown in the devices described herein. In this embodiment, cells, such as tumor cells, established cell lines such as HeLa cells or NHN 3T3 fibroblasts or tissue biopsies, are grown in or on the elastomeric substrate and the cells are contacted with the analyte. Any appropriate analyte-specific marker can be evaluated in order to determine the effect of the analyte on the cells. For instance, if the analyte is a drug being tested for safety, cell growth or death may be monitored. If the drug is tested for efficacy, for example as an anticarcinogen and the cells are a specific cancer type, such as cells derived from a biopsy or resection of a patient's tumor, the cells can be monitored for growth and/or cell death. Other markers include expression of a desired gene or production of a specific gene product, for example and without limitation, a cytokine. Other variations on the described analyte testing would be readily apparent to one of skill in the art. As used herein, “analyte” refers to any compound or composition that can be tested for phenotypic or genotypic response from the cells. The analyte can be, without limitation, a chemical, a drug, a biologic, such as a nucleic acid or protein, a virus, a bacterium, a fungal cell, a eukaryotic cell, a cell lysate or a suspected carcinogen.

The device described herein represents a significant resource for simultaneously modulating multiple environmental parameters (mechanics, scaffolding, and chemistry) with concurrent measurements of cell responses using biological staining and imaging. With this device, we can explore the local activation of biochemical responses, spatial distribution of the focal adhesion complexes, and examine transmembrane proteins involved in mechanotransduction. The ability to apply mechanical stimulation with the device in a three-dimensional configuration, while simultaneously controlling the scaffolding and chemistry, greatly enhances our understanding of cellular responses. This research has lead to significant advances in establishing a multi-faceted in vitro technique, which mimics in vivo environments over a wide range of applications. These observations, in turn, will improve our knowledge of cellular and molecular functions, and will provide useful insights in related fields, including in vitro diagnostics, biomechanics, tissue engineering, and drug discovery.

### EXAMPLES

### Example 1

### Elastomeric Substrate with Extracellular Matrix Mimetic Coating

Membrane Preparation

An elastomeric substrate was custom fabricated for use with the PDMS flexible material to mechanically stimulate single cells. First, the PDMS substrates were prepared using Sylgard 184 (Dow Corning) with a key constraint of controlling thickness, which correlated directly to the applied strain during pressurized deformation, essentially as shown in FIG. 1G. The circular dimension of the PDMS for equibiaxial stretching was 6-cm diameter. Reproducible thicknesses were produced by distributing PDMS evenly over a flat substrate (e.g. petri dish or a silicon wafer), allowing bubbles to naturally dissipate from the fluid and then polymerizing it through concentrated application of heat for 20 minutes. The substrate was sterilized with ethanol and subsequently rinsed with PBS three times before use. Next, 150 μL of 10 μg/mL human fibronectin (BD Biosciences) solubilized in PBS was placed on the membrane and after 60 minutes of incubation at 23° C., the substrate was rinsed with PBS prior to cell seeding.

Cell Culture

NIH 3T3 fibroblast cells were cultured with Dulbecco's Modified Eagle's Medium supplemented by 10% calf serum, glutamine (0.3 mg/ml), penicillin (100 U/ml), and streptomycin (100 μg/ml), and 20 mM N-2-hydroxyethylpiperazine-N′-2-ethanesulfonic acid at pH of 7.4 under 5% carbon dioxide. Before seeding on the flexible membranes, the cells were washed once with phosphate buffered saline (PBS) and then exposed to trypsin-ethylenediaminetetraacetate (EDTA). After dissociation from the tissue culture plates, the cells were counted and then cultured onto the flexible membranes of polydimethylsiloxane (PDMS) at approximately 4300 cells/cm². The cells were subsequently incubated for at least 6 hours to allow for attachment and spreading prior to mechanical stimulation.

Visualization of Cell Morphology and Fibronectin

The direct morphology of single cells and indirect immunofluorescence localization of the ECM component, fibronectin (FN), was accomplished with an inverted microscope. For examination of cell morphology and fibronectin, the fibroblasts were fixed in 4% paraformaldehyde diluted in PBS and then rinsed with PBS three times. We mounted the sample on a #1 borosilicate coverslide with Fluoromount-G and the fibronectin was conjugated to a tetramethylrhodamine isothiocyanate fluorophore to allow for microscopic visualization. Epi-fluorescence and differential interference contrast (DIC) microscopy was performed on a Zeiss Axiovert with an Insight digital camera and NIH Image analysis software.

In this Example, NIH 3T3 fibroblasts were cultured on flexible substrates for the PECS system coated with varying concentrations of fibronectin (10 ng/mL, 100 ng/mL, 1 μg/mL, 10 μg/mL, 100 μg/mL) to optimize first the conditions for attachment and spreading. An effective range of 100 ng/mL to 100 μ/mL was determined, with an optimal concentration of about 10 μg/mL for the PDMS membrane. When the fibronectin concentration was outside the effective range, cells did not attach and spread. Cells with the optimal concentration attached and spread on the membrane as shown in FIG. 4A.

In an additional experiment, the adherent molecular regions were modulated through the addition of spatially constrained amounts of fibronectin 610 on membrane 600, as shown in FIGS. 4B and 4C. This revealed the necessity for this ECM coating for cell spreading. After cell attachment and spreading on the elastomeric substrate, the membrane was rinsed with PBS and manually clamped into the equibiaxial cell stretcher, substantially as shown and described in relation to FIGS. 1A through 1G, for deformation with external applied pressure. This system was custom fabricated to allow for an aqueous environment on the upper surface requisite for cell culture. Prior to the mechanical stimulation, the media was replaced with CO₂-Independent Medium, commercially available from Invitrogen of Carlsbad, Calif., supplemented similarly to the growth media and modulated the temperature of the media through a heat source to maintain 37° C. for the duration of the experiment.

The deformation of the elastomeric substrate was precisely controlled with a pressure regulator. After cells were cultured on the upper surface of the membrane to form an adherent monolayer and introduced into the device, a controlled increase in the pressure on the lower surface of the membrane created an associated equibiaxial stretching due to the fixed displacement boundary conditions on the periphery of the clamped membrane. This constraint confined the perimeter of the flexible membrane due to the circular clamp configuration, which could be adjusted to precisely control the displacement profile and therefore, the deformation of the flexible membrane, thereby mechanically straining the cells that were connected to the substrate through the ECM.

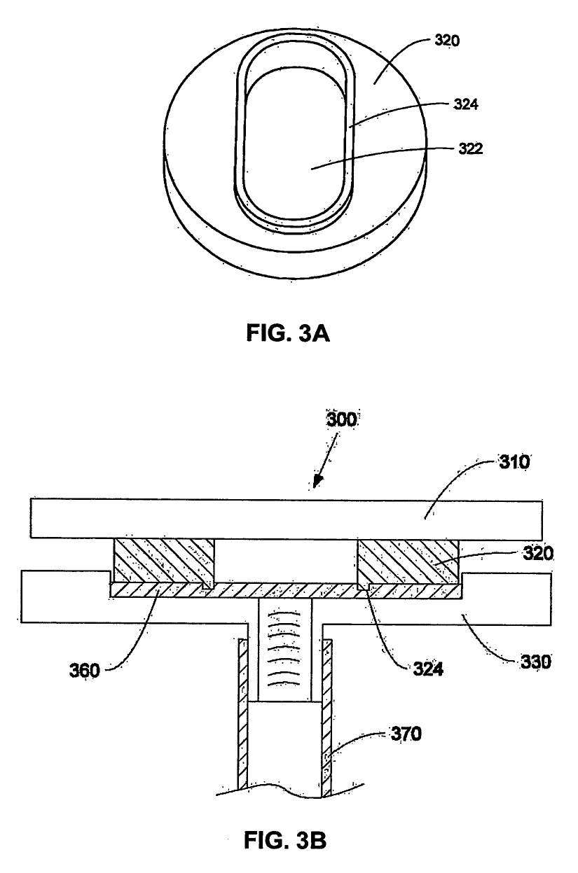

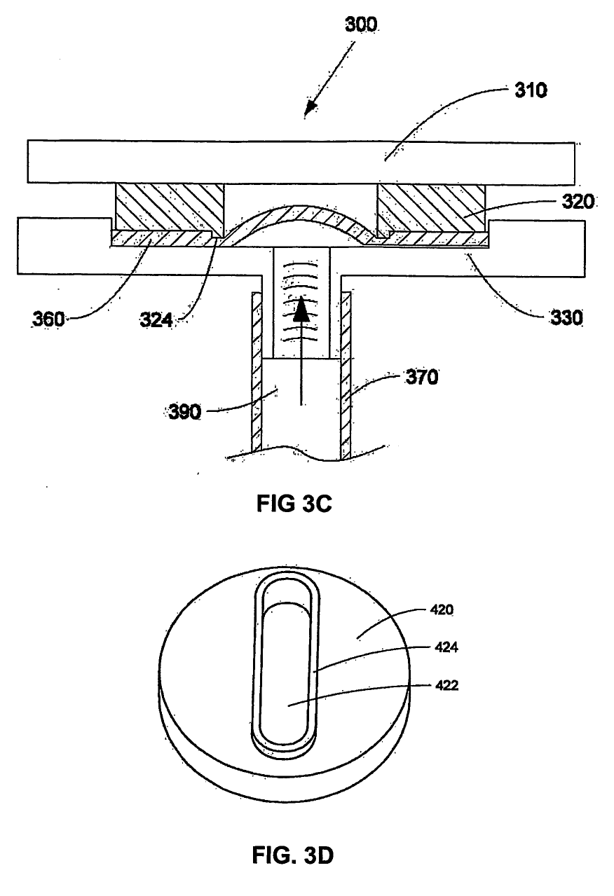

To determine the relationship between pressure on the substrate and the amount of strain induced in the substrate, theoretical calculations were undertaken. Using an equation designed for thin film bulge tests we approximated the membrane behavior as a function the vertical displacement up to 1% strain. By comparing the actual membrane deformation, the method that most accurately characterized the strain behavior of the PDMS membrane was determined. The bulge test approximation is provided in equation I.

$$σ = 2 \frac{{b}^{2}}{3 {a}^{2}} \qquad ( I )$$

where σ was the strain, and a and b were the major and minor dimensions of the ellipse. The first strain calculation was made by assuming that the substrate would form an ellipsoid shape as the pressure increased, and the exact summation for an ellipse is provided in equations II and III:

$$\begin{matrix}P = {\sum }_{n = 0}^{∞} \frac{- {ⅇ}^{2 n}}{2 n - 1} {( \frac{( 2 n ) !}{{( {2}^{n} n ! )}^{2}} )}^{2} & ( II ) \\ e = \sqrt{1 - \frac{{b}^{2}}{{a}^{2}}} & ( III )\end{matrix}$$

where P was the perimeter and e was the eccentricity. Although this method was exact, it was challenging to implement because the series converged slowly. An alternate approximation is Ramanujan's ellipse approximation provided in equation IV:
P=π└3(a+b)−\sqrt{(3a+b)(a+3b)}┘ (IV)
Ramanujan's approximation is accurate for any ellipse except those where the eccentricity approaches 1 although this resulted in a minimal error of −0.4. These solutions assumed elliptical deformation of the membrane as it was pressurized A triangular approximation indicating a linear deformation as a limiting case was made as well, as shown in FIG. 5A.

To correlate these theoretical calculations with experimental results, a laser interferometer was used to measure the vertical displacement of the center point of the circular PDMS membrane. Due to the edge constraints of the system, the midpoint of the circular membrane reflected the region with the maximum vertical displacement. These experiments revealed a relatively linear relationship between pressure and vertical displacement, as shown in FIG. 5B; these further were verified using the approximate solutions for the deformation.

From this information on the membrane deformation, adherent fibroblasts were confidently subjected to strains of up to 5% for 30 minutes using this system. The cells were then fixed for morphological examination with the eventual proposition of surveying the cytoskeletal and focal adhesion complex organization as the effects of stress have been observed to affect cell morphology and actin cytoskeleton alignment when individual cells sense and respond to the application of external force (Sumpio, B. E., Banes, A. J., Levin, L. G. & Johnson, G., Jr. (1987) J Vasc Surg 6, 252-6.).

Further experiments were conducted on single cells under a range of strains from 0 to 5%. The strain also was varied temporally from 0 to 60 minutes. We have thus demonstrated the ability to controllably apply three-dimensional mechanical stimulation on single cells through these ECM-coated elastomeric membranes.

### Example 2

### Three-Dimensional Elastomeric Cell Growth Substrates

Cell Culture.

Before seeding on the flexible membranes, the cells were washed once with PBS and then exposed to trypsin-EDTA for 3 minutes. After dissociation from the tissue culture plates, the cells were counted and then cultured onto the flexible membranes of PDMS at approximately 4300 cells/cm². The cells were subsequently incubated for at least 6 hours to allow for attachment and spreading prior to mechanical stimulation. The medium was replaced, while a thermostatically controlled heat source maintained the sample temperature at 37° C. for the duration of the experiment, which allowed for a controlled aqueous environment, required for cell culture on the upper surface prior to mechanical stimulation.

Membrane Coating for Cell Attachment.

The membrane was coated with 150 μl of 10 μg/mL human fibronectin (FN) dissolved in phosphate-buffered saline solution (PBS) and, after 60 min of incubation at 23° C., the substrate was seeded with NIH 3T3 fibroblasts. Coatings of varying concentrations of fibronectin were applied in the device to optimize the conditions for attachment and spreading, 10 μg/ml being optimal, though the effective range of fibronectin concentrations was broader, as described above. At suboptimal fibronectin concentrations, the cells remained in suspension, whereas at optimal concentrations they attached to, and spread over the membrane. After cell attachment and spreading on the elastomeric substrate, the membrane was rinsed with PBS and manually clamped in the equibiaxial cell stretcher for deformation by externally applied pressure.

Microscope Visualization.

The direct morphology of single cells was observed with an Axiovert inverted Zeiss microscope. For examination of cell morphology, the fibroblasts were fixed in 4% paraformaldehyde diluted in PBS and then permeabilized in 0.1% TritonX-100 diluted in PBS. The cells were immunofluorescently labeled with phalloidin and Dapi to observe actin filaments and the nucleus. The sample was mounted on a #1 borosilicate coverslide with Fluoromount-G. Differential interference contrast and epi-fluorescence microscopy were performed with an Insight digital camera and NIH Image analysis software with dapi and fluorescein isothiocyanate filter sets under 10×, 20×, and 63× (1.4 numerical aperture) objectives.

### RESULTS AND DISCUSSION

The described membrane integrates fabrication and molecular technologies to generate cellular patterns which mimic ECM-cell interactions in vivo, and enable the application of three-dimensional mechanical stimuli. First, we modulated the adherent molecular regions through the application of spatially constrained areas of fibronectin. To analyze the structural form of cell regulation without the limitations of overlaying conventional tissue-culture dishes with varying amounts or distributions of non-adhesive blocking agent, we built patterns of adhesive regions with size, shape, and position defined on the micron scale, surrounded by non-adhesive boundary regions established with Pluronic block copolymers or bovine serum albumin. The substrates were coated with a saturating density of fibronectin, and the cells were placed on these surfaces in a medium devoid of serum to prevent the deposition of extracellular matrix molecules on the non-adhesive surfaces. We maintained control over the size and shape of the adhesive surface, as well as controlled the extent to which the cells could distend. When cells adhered to the extracellular matrix-coated region, they spread over fixed extracellular matrix anchors to cover the adhesive region, stopping when their periphery had reached the non-adhesive boundary on the PDMS. As a result, they changed their morphology to assume the geometry of their molecularly patterned substrate as shown in FIG. 4C and as was described above, in Example 1. This allowed us to analyze the effects of varying cell populations and single-cell sizes and shapes on cell functions (e.g., proliferation, apoptosis, differentiation) in combination with three-dimensional mechanical stimulation with the apparatus. This was a critical step, since local changes in cell-ECM interactions heavily influence growth differentials that drive pattern formation and the structural basis of morphogenic regulation in vivo.

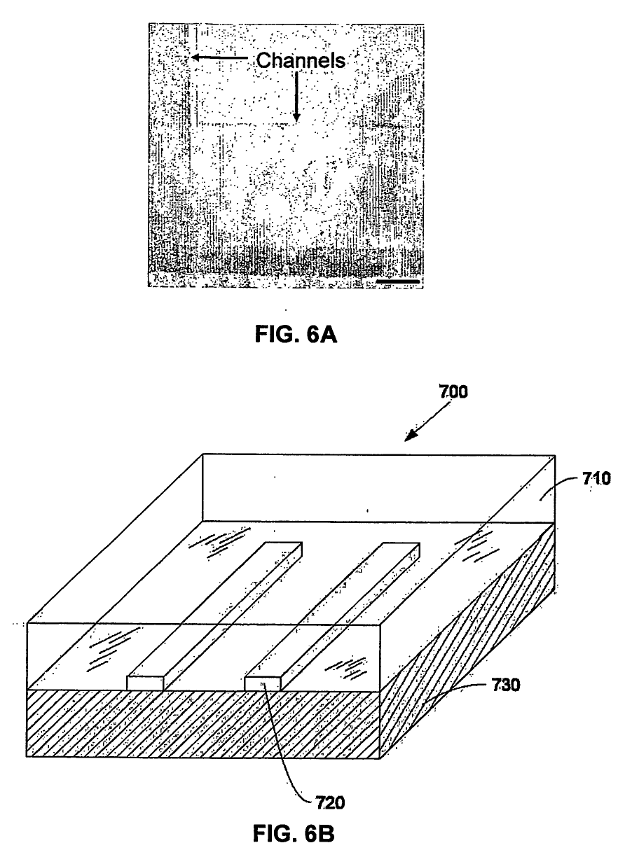

By controlling the membrane architecture with fabrications techniques, three-dimensional networks were integrated with the PreCS device to create mechanical stimulation environments mimicking in vivo conditions. This is useful in a multitude of physiological environments where heterogeneous mechanical and cellular scaffolding is required, such as in the simulation of vasculature for tissue-engineering applications. Enclosed geometries inside the PDMS were fabricated through lithographic techniques. Rectangular channels were created using negative photoresist layers on silicon wafers. Patterns were printed onto transparent masks, after which the wafers were exposed to ultraviolet light with the mask adherent to the resist layer. After exposing and developing the photoresist, the remaining photoresist was used as a positive mold and then the PDMS was placed on the surface of the wafer and thermally cured. The resultant membrane containing engineered surface channels is shown in FIG. 6A. The membrane thickness was defined by the rotational speed of the wafer during introduction of the PDMS. In relation to FIG. 6B, structure 700 shown schematically in that Figure is prepared by inverting attaching membrane 710, including exposed channel 720, onto a second membrane 730 and thermally laminating the two membranes 710 and 730. The size of the channels could be controlled in three dimensions down to single microns. Cells were cultured in these embedded membrane networks, as well as in exposed channels lacking an upper membrane, as shown in FIG. 6C. The cells attached themselves to one or more supporting surfaces of the channels, exposing them to three-dimensional mechanical stimulation and scaffolding support. The device also allowed cellular immunostaining, which we used to examine the cytoskeletal and focal adhesion complex organization (FIG. 7), as the effects of stress affect morphology and actin cytoskeleton alignment when individual cells sense and respond to the application of external force.

To study more complex, anisotropic mechanical stimulation, we enhanced the performance of the described device by redefining the boundary constraints of the system as well as creating membranes with materials of varying elastic moduli or topography. The circular clamping constraints were modified to obtain an elliptical configuration and impose gradients of strain using masks essentially as shown in FIGS. 3A, 3D and 3E. The major axis of the elliptical membrane remained constant while the minor axis was decreased in ratios of 2, 4, and 8. This imposed a curvature that was greater over the length of the major than the minor axis. The minor-axis length was decreased, therefore, the strain, ε of the membrane and associated cells approached uniaxial conditions (ε_{minor axis}>>ε_{major axis}). This made it possible to impose varying mechanical deformation profiles on cells cultured on these membranes, over the surface of the membrane. The methodology was expanded to impose heterogeneous three-dimensional deformation by varying the elastomeric properties of the membrane in defined domains throughout the substrate.

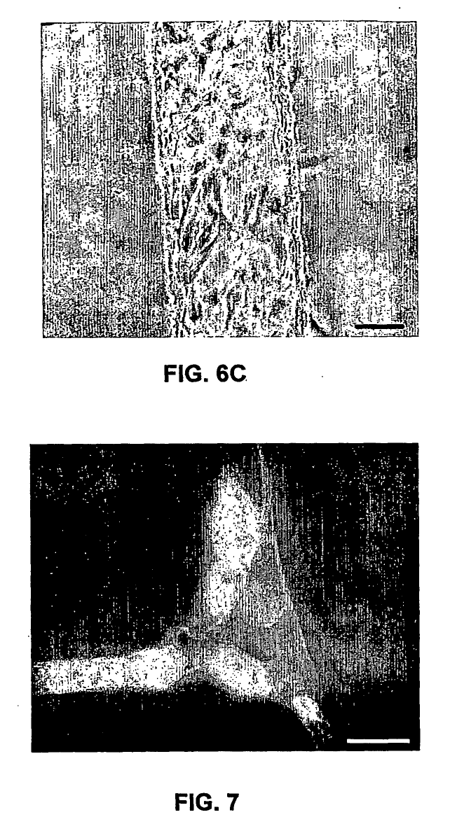

Variations of the elastic moduli through the membrane were introduced through the infusion of materials with higher elastic moduli than the PDMS, including a woven nylon matrix (E_{PDMS}=2×10⁵ Pa, E_{nylon}=2×10⁹ Pa). Defined stress-strain profiles were developed through the application of embedding materials and varying surface topologies over the diameter of the film. Through repeating patterns of materials at the micrometer scale embedded in the polymer, topologies were produced with strain gradients across the width of the membrane, as shown in FIG. 8. Alternately, the thickness profile of the membrane and the resultant strain profile were controlled by membrane topologies through a time-delay thermal curing cycle. The variations in the PDMS surface produced complex deformation of the cells (data not shown).

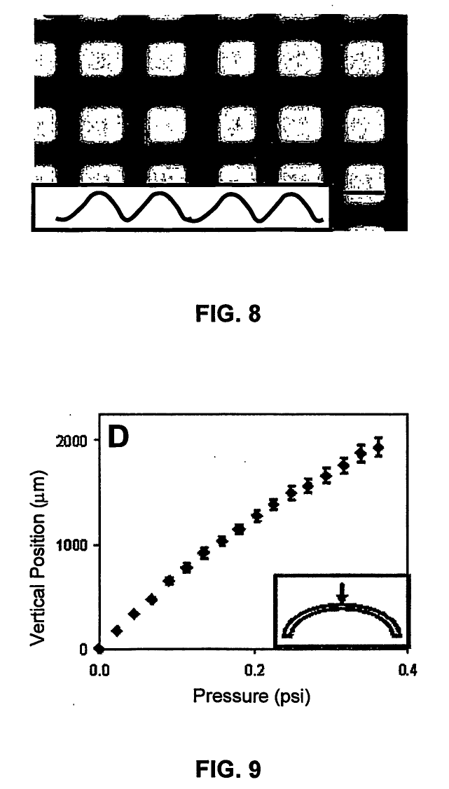

To quantify the strain applied by the device, the vertical displacement of the PDMS membrane was first measured optically by non-contact laser interferometry at the center point of the circular PDMS membrane. A retro-reflective coating was applied to the surface of the PDMS membrane to increase the signal-to-noise ratio from the optical head. The displacement was measured with a Michelson interferometer (Polytec; GmbH, Waldbronn, Germany), which employs fiber-optic bundles to control the paths of the reference and target laser beams. Due to the edge constraints and circular nature of the equidistant constrained system, the midpoint of the membrane reflected the region with the maximum vertical displacement (from 100 μm to 2 cm). Mathematical techniques are needed to precisely describe the strain on a system of cells under the influence of a pressure-driven membrane. The relationship between pressure and strain in the substrate was characterized through equation V, which revealed that circularly clamped elastic membranes behave like spherical membranes when exposed to a pressure exceeding a particular stress threshold. The shape of the deformation is approximated by the surface of a sphere where the bulge-test equation is valid. If a is the diameter of the sample and h is the vertical deflection, an approximately linear relationship between applied pressure and vertical displacement is revealed over a range of strains up to 25% (FIG. 9). The stress σ is:

$$σ = \frac{2}{3} {( \frac{h}{a} )}^{2} . \qquad ( V )$$

The threshold of this thin-film approximation is correlated with the ratio of the change in vertical displacement of the film to the pre-strain thickness of the film. This must be >10 for a 500-μm thick membrane; our displacements were ≦5 mm.

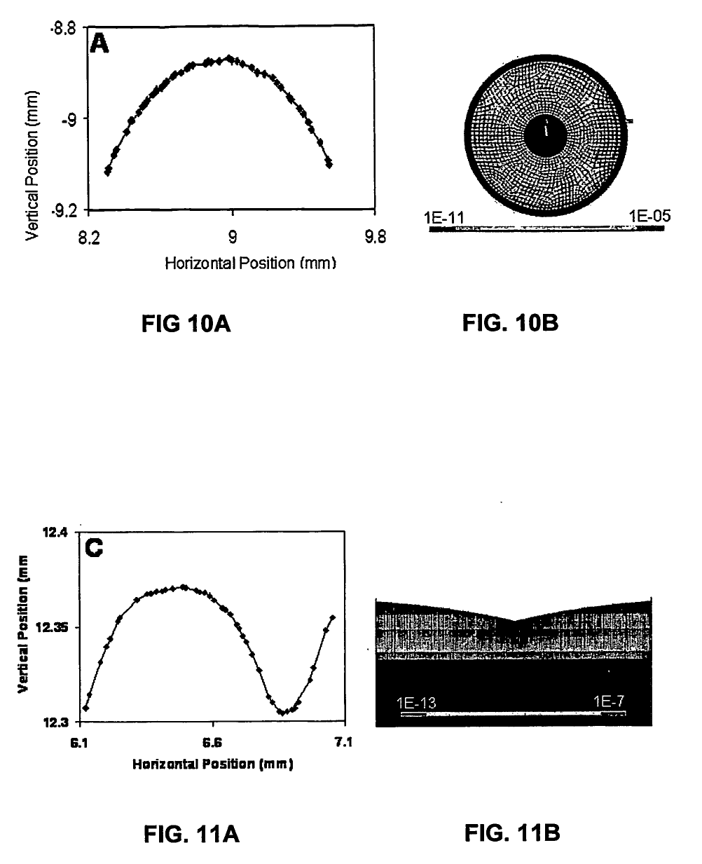

The membrane was exposed to a constant-pressure environment. Nevertheless, with elastic-modulus mismatches, the induced strains varied across the section. This displacement was measured with a MicroVal Coordinate Measuring Machine (Brown & Sharpe; North Kingstown, R.I.), which is accurate to 10 μm for the x, y, and z coordinates of an orthogonal Cartesian-coordinate system. As the surface of the membrane was traversed, the vertical deflection followed an essentially spherical configuration with the deflection slope increasing as the radial distance from the center of the membrane increased (FIG. 10A). The deformation of the membrane was also modeled using finite-element software (ANSYS®; Canonsburg, Pa.) using clamped boundary conditions at the periphery of an elastic membrane modeled with shell elements. The elastic modulus of PDMS was used as a homogeneous material property with a constant pressure from below the basal side of a multi-layered element system. The resulting displacements in a circularly constrained membrane deformation system revealed the deformation due to the pinned periphery constraint on the membrane, consistent with our experimental observations (FIG. 10B). Thus the surface profile captures the essence of the embedded composite material. Experimental and simulation results when embedding a thin rectangular section of nylon matrix at the interior of the membrane reveals a divergence in deformation from the previously described spherical deformation of the homogeneous membrane (FIGS. 11A and 11B). The device described herein device is able to apply a bifurcation of strain due to composite formed from the embedded bar within PDMS.

We claim:

1. A cell growth apparatus comprising a cell growth chamber having an interior side and an exterior side and comprising a wall and a base defining an interior volume, the cell growth chamber comprising an elastomeric growth substrate comprising an elastomeric membrane of a first material that comprises a first portion having a first elasticity and a second portion having a second elasticity.

2. The apparatus of claim 1, wherein at least a portion of the base of the cell growth chamber consists of the elastomeric growth substrate.

3. The apparatus of claim 2, further comprising a secondary chamber in fluid connection with and partially defined by an exterior side of the elastomeric growth substrate, the secondary chamber comprising an opening having a fitting for a pipe or tube.

4. The apparatus of claim 3, further comprising a pump in fluid communication with the secondary chamber.

5. The apparatus of claim 1, wherein the elastomeric membrane has a portion of a first thickness, having a first elasticity, and a portion of a second thickness, having a second elasticity.

6. The apparatus of claim 1, wherein a second material having a different elasticity than the first material is embedded within or attached to the elastomeric membrane.

7. The apparatus of claim 6, wherein the second material is one of a polymer, a metal, a ceramic and a fabric.

8. The apparatus of claim 7, wherein the second material is a nylon mesh.

9. The apparatus of claim 7, wherein the second material is a stainless steel mesh.

10. The apparatus of claim 1, wherein the substrate further comprises one or more additional elastomeric layers, at least one of which is attached to the elastomeric membrane.

11. The apparatus of claim 10, wherein one or more of the additional elastomeric layer is biodegradable.

12. The apparatus of claim 11, wherein the biodegradable layer comprises a poly(glycerol-sebacate) polymer.

13. The apparatus of claim 1, wherein the interior side of the elastomeric membrane is partially or fully coated with an extracellular matrix-mimetic.

14. The apparatus of claim 13, wherein the extracellular matrix mimetic is selected from the group consisting of fibronectin, vitronectin, collagen, laminin, poly(lactide) , poly(lactide-co-glycolide) and a self-complementary oligopeptide matrix.

15. The apparatus of claim 13, where in the extracellular matrix mimetic is fibronectin.

16. The apparatus of claim 13, wherein the extracellular matrix mimetic partially coats the interior side of the elastomeric membrane.

17. The apparatus of claim 16, further comprising an adhesion inhibitor covering parts of the interior side of the elastomeric membrane not covered by the extracellular matrix mimetic.

18. The apparatus of claim 17, wherein the adhesion inhibitor is one of bovine serum albumin and a poly(ethylene oxide)/polypropylene oxide)/poly(ethylene oxide) triblock polymer.

19. The apparatus of claim 1, wherein the first portion has a first elastic modulus and the second portion has a second elastic modulus.

20. The apparatus of claim 1, wherein the membrane comprises one or more internal passageways.

21. The apparatus of claim 1, wherein the membrane comprises one or more engineered structural formations.

22. The apparatus of claim 21, wherein the engineered structural formation is one of a surface groove and a passageway within the membrane.

23. The apparatus of claim 22, wherein the surface groove or passageway within the membrane has a diameter of less than 100μ.

24. The apparatus of claim 22, wherein the membrane comprises an internal passageway that opens into the interior volume.

25. The apparatus of claim 22 wherein the passageway is coated with an extracellular matrix mimetic.

26. The apparatus of claim 1, wherein the elastomeric membrane is biodegradable.

27. The apparatus of claim 26, wherein- the biodegradable membrane comprises a poly(glycerol-sebacate) polymer.

28. The apparatus of claim 1, wherein the wall is annular.

29. The apparatus of claim 1, wherein the wall is ellipsoid.

30. The apparatus of claim 1, wherein at least a portion of the substrate is coated with an adhesion promoter.

31. An elastomeric cell growth substrate comprising an elastomeric membrane of a first material that comprises a first portion having a first elasticity and a second portion having a second elasticity.

32. The substrate of claim 31, wherein the elastomeric membrane has a portion of a first thickness, having a first elasticity, and a portion of a second thickness, having a second elasticity.

33. The substrate of claim 31, wherein a second material having a different elasticity than the first material is embedded within or attached to the elastomeric membrane.

34. The substrate of claim 33, wherein the second material is one of a polymer, a metal, a ceramic and a fabric.

35. The substrate of claim 34, wherein the second material is a nylon mesh.

36. The substrate of claim 34, wherein the second material is a stainless steel mesh.

37. The substrate of claim 31, wherein the substrate further comprises one or more additional elastomeric layers, at least one of which is attached to the elastomeric membrane.

38. The substrate of claim 37, wherein one or more of the additional elastomeric layer is biodegradable.

39. The substrate of claim 38, wherein the biodegradable layer comprises a poly(glycerol-sebacate) polymer.

40. The substrate of claim 31, wherein the interior side of the elastomeric membrane is partially or fully coated with an extracellular matrix-mimetic.

41. The substrate of claim 40, wherein the extracellular matrix mimetic is selected from the group consisting of fibronectin, vitronectin, collagen, laminin, poly(lactide), poly(lactide-co-glycolide) and a self-complementary oligopeptide matrix.

42. The substrate of claim 40, where in the extracellular matrix mimetic is fibronectin.

43. The substrate of claim 40, wherein the extracellular matrix mimetic partially coats the interior side of the elastomeric membrane.

44. The substrate of claim 43, further comprising an adhesion inhibitor covering parts of the interior side of the elastomeric membrane not covered by the extracellular matrix mimetic.

45. The substrate of claim 44, wherein the adhesion inhibitor is one of bovine serum albumin and a poly(ethylene oxide)/poly(propylene oxide)/poly(ethylene oxide) triblock polymer.

46. The substrate of claim 31, wherein the first portion has a first elastic modulus and the second portion has a second elastic modulus.

47. The substrate of claim 31, wherein the membrane comprises one or more internal passageways.

48. The substrate of claim 31, wherein the membrane comprises one or more engineered structural formations.

49. The substrate of claim 48, wherein the engineered structural formation is one of a surface groove and a passageway within the membrane.

50. The substrate of claim 49, wherein the surface groove or passageway within the membrane has a diameter of less than 100μ.

51. The substrate of claim 49, wherein the membrane comprises an internal passageway that opens into the interior volume.

52. The substrate of claim 51, wherein the passageway is coated with an extracellular matrix mimetic.

53. The substrate of claim 31, wherein the elastomeric membrane is biodegradable.

54. The substrate of claim 53, wherein the biodegradable membrane comprises a poly(glycerol-sebacate) polymer.

55. The substrate of claim 31, wherein at least a portion of the substrate is coated with an adhesion promoter.

56. A cell growth apparatus comprising a cell growth chamber having an interior side and an exterior side and comprising a wall and a base defining an interior volume, the cell growth chamber comprising an elastomeric growth substrate comprising an elastomeric membrane of a first material having an interior side and an exterior side, wherein the elastomeric membrane is at least partially coated with an extracellular matrix-mimetic.

57. The apparatus of claim 56, wherein the membrane comprises a first portion having a first elasticity and a second portion having a second elasticity.

58. The apparatus of claim 56, wherein at least a portion of the base of the cell growth chamber consists of the elastomeric growth substrate.

59. The apparatus of claim 58, further comprising a secondary chamber in fluid connection with and partially defined by an exterior side of the elastomeric growth substrate, the secondary chamber comprising an opening having a fitting for a pipe or tube.

60. The apparatus of claim 59, further comprising a pump in fluid communication with the secondary chamber.

61. The apparatus of claim 56, wherein the elastomeric membrane has a portion of a first thickness, having a first elasticity, and a portion of a second thickness, having a second elasticity.

62. The apparatus of claim 56, wherein a second material having a different elasticity than the first material is embedded within or attached to the elastomeric membrane.

63. The apparatus of claim 62, wherein the second material is one of a polymer, a metal, a ceramic and a fabric.

64. The apparatus of claim 63, wherein the second material is a nylon mesh.

65. The apparatus of claim 63, wherein the second material is a stainless steel mesh.

66. The apparatus of claim 56, wherein the substrate further comprises one or more additional elastomeric layers, at least one of which is attached to the elastomeric membrane.

67. The apparatus of claim 66, wherein one or more of the additional elastomeric layer is biodegradable.

68. The apparatus of claim 67, wherein the biodegradable layer comprises a poly(glycerol-sebacate) polymer.

69. The apparatus of claim 56, wherein the extracellular matrix mimetic is selected from the group consisting of fibronectin, vitronectin, collagen, laminin, poly(lactide), poly(lactide-co-glycolide) and a self-complementary oligopeptide matrix.

70. The apparatus of claim 69, where in the extracellular matrix mimetic is fibronectin.

71. The apparatus of claim 56 wherein the first portion has a first elastic modulus and the second portion has a second elastic modulus.

72. The apparatus of claim 56, wherein the membrane comprises one or more internal passageways.

73. The apparatus of claim 56, wherein the membrane comprises one or more engineered structural formations.

74. The apparatus of claim 73, wherein the engineered structural formation is one of a surface groove and a passageway within the membrane.

75. The apparatus of claim 74, wherein the surface groove or passageway within the membrane has a diameter of less than 100μ.

76. The apparatus of claim 75, wherein the membrane comprises an internal passageway that opens into the interior volume.

77. The apparatus of claim 76, wherein the passageway is coated with an extracellular matrix mimetic.

78. The apparatus of claim 56, wherein the extracellular matrix mimetic partially coats the interior side of the elastomeric membrane.

79. The apparatus of claim 78, further comprising a adhesion inhibitor covering parts of the interior side of the elastomeric membrane not covered by the extracellular matrix mimetic.

80. The apparatus of claim 79, wherein the adhesion inhibitor is one of bovine serum albumin and a poly(ethylene oxide)/poly(propylene oxide)/poly(ethylene oxide) triblock polymer.

81. The apparatus of claim 56, wherein the elastomeric membrane is biodegradable.

82. The apparatus of claim 81, wherein the biodegradable membrane comprises a poly(glycerol-sebacate) polymer.

83. The apparatus of claim 56, wherein the wall is annular.

84. The apparatus of claim 56, wherein the wall is ellipsoid.

85. The apparatus of claim 56, wherein at least a portion of the substrate is coated with an adhesion promoter.

86. A cell growth substrate, comprising an elastomeric membrane of a first material that is at least partially coated with an extracellular matrix-mimetic.

87. The substrate of claim 86, wherein the membrane comprises a first portion having a first elasticity and a second portion having a second elasticity.

88. The substrate of claim 86, wherein the elastomeric membrane has a portion of a first thickness, having a first elasticity, and a portion of a second thickness, having a second elasticity.

89. The substrate of claim 86, wherein a second material having a different elasticity than the first material is embedded within or attached to the elastomeric membrane.

90. The substrate of claim 89, wherein the second material is one of a polymer, a metal, a ceramic and a fabric.

91. The substrate of claim 90, wherein the second material is a nylon mesh.

92. The substrate of claim 90, wherein the second material is a stainless steel mesh.

93. The substrate of claim 86, wherein the substrate further comprises one or more additional elastomeric layers, at least one of which is attached to the elastomeric membrane.

94. The substrate of claim 93, wherein one or more of the additional elastomeric layer is biodegradable.

95. The substrate of claim 94, wherein the biodegradable layer comprises a poly(glycerol-sebacate) polymer.

96. The substrate of claim 86, wherein the extracellular matrix mimetic is selected from the group consisting of fibronectin, vitronectin, collagen, laminin, poly(lactide), poly(lactide-co-glycolide) and a self-complementary oligopeptide matrix.

97. The substrate of claim 96, where in the extracellular matrix mimetic is fibronectin.

98. The substrate of claim 86, wherein the first portion has a first elastic modulus and the second portion has a second elastic modulus.

99. The substrate of claim 86, wherein the membrane comprises one or more internal passageways.

100. The substrate of claim 86, wherein the membrane comprises one or more engineered structural formations.

101. The substrate of claim 100, wherein the engineered structural formation is one of a surface groove and a passageway within the membrane.

102. The substrate of claim 101, wherein the surface groove or passageway within the membrane has a diameter of less than 100μ.

103. The substrate of claim 101, wherein the membrane comprises an internal passageway that opens into the interior volume.

104. The substrate of claim 103, wherein the passageway is coated with an extracellular matrix mimetic.

105. The substrate of claim 86, wherein the extracellular matrix mimetic partially coats the interior side of the elastomeric membrane.

106. The substrate of claim 105, further comprising an adhesion inhibitor agent covering parts of the interior side of the elastomeric membrane not covered by the extracellular matrix mimetic.

107. The substrate of claim 106, wherein the adhesion inhibitor is one of bovine serum albumin and a poly(ethylene oxide)/poly(propylene oxide)/poly(ethylene oxide) triblock polymer.

108. The substrate of claim 86, wherein the elastomeric membrane is biodegradable.

109. The substrate of claim 108, wherein the biodegradable membrane comprises a poly(glycerol-sebacate) polymer.

110. The apparatus of claim 86, wherein at least a portion of the substrate is coated with an adhesion promoter.

111. A method of producing an elastomeric cell growth substrate, comprising coating at least a portion of an elastomeric membrane with an extracellular matrix mimetic.

112. The method of claim 111, wherein the extracellular matrix mimetic is selected from the group consisting of fibronectin, vitronectin, collagen, laminin, poly(lactide), poly(lactide-co-glycolide) and a self-complementary oligopeptide matrix.

113. The method of claim 111, where in the extracellular matrix mimetic is fibronectin.

114. The method of claim 111, further comprising coating at least a portion of the elastomeric membrane with an adhesion inhibitor.

115. The method of claim 114, wherein the adhesion inhibitor is bovine serum albumin.

116. The method of claim 114, wherein the adhesion inhibitor is a poly(ethylene oxide)/poly(propylene oxide)/poly(ethylene oxide) triblock polymer.

117. The method of claim 111, wherein the membrane has a first portion having a first elasticity and a second portion having a second elasticity.

118. The method of claim 117, wherein the first portion has a first elastic modulus and the second portion has a second elastic modulus.

119. The method of claim 117, wherein the membrane has portions of differing thickness.

120. The method of claim 117, wherein a material of a different elastic modulus than that of the membrane is embedded within or attached to the membrane.

121. The method of claim 120, wherein the material is one of a nylon mesh and a stainless steel mesh.

122. The method of claim 117, wherein the membrane comprises one or more internal passageways.

123. The method of claim 111, wherein the membrane comprises one or more engineered structural formations.

124. The method of claim 123, wherein the engineered structural formation is one of a surface groove and a passageway within the membrane.

125. The method of claim 124, wherein the surface groove or passageway within the membrane has a diameter of less than 100μ.

126. The method of claim 123, wherein the membrane is prepared by curing an elastomeric polymer in a mold containing a form defining the engineered structural formation.

127. The method of claim 126, wherein the form defining the engineered structural formation is a silicon wafer comprising a patterned photoresist layer defining the engineered structural formation.

128. The method of claim 126, comprising pouring PDMS over a silicon wafer comprising a patterned photoresist layer defining the engineered structural formation and heat curing the PDMS.

129. The method of claim 126, wherein the engineered structural formation is a channel.

130. The method of claim 123, wherein a second elastomeric layer is attached to the membrane.

131. The method of claim 130, wherein the engineered structural formation is a groove and the second elastomeric layer is aligned over the groove to form a passageway.

132. A method of producing an elastomeric cell growth substrate, comprising, preparing an elastomeric membrane of a first material that comprises a first portion having a first elasticity and a second portion having a second elasticity.

133. The method of claim 132, comprising coating at least a portion of the elastomeric membrane with an extracellular matrix mimetic.

134. The method of claim 132, wherein the extracellular matrix mimetic is selected from the group consisting of fibronectin, vitronectin, collagen, laminin, poly(lactide), poly(lactide-co-glycolide) and a self-complementary oligopeptide matrix.

135. The method of claim 133, where in the extracellular matrix mimetic is fibronectin.

136. The method of claim 132, further comprising coating at least a portion of the elastomeric membrane with an adhesion inhibitor.

137. The method of claim 136, wherein the adhesion inhibitor is bovine serum albumin.

138. The method of claim 136, wherein the adhesion inhibitor is a poly(ethylene oxide)/poly(propylene oxide)/poly(ethylene oxide) triblock polymer.

139. The method of claim 132, wherein the first portion has a first elastic modulus and the second portion has a second elastic modulus.

140. The method of claim 132, wherein the membrane has portions of differing thickness.

141. The method of claim 132, wherein a material of a different elastic modulus than that of the membrane is embedded within or attached to the membrane.

142. The method of claim 141, wherein the material is one of a nylon mesh and a stainless steel mesh.

143. The method of claim 132, wherein the membrane comprises one or more internal passageways.

144. The method of claim 132, wherein the membrane comprises one or more engineered structural formations.

145. The method of claim 144, wherein the engineered structural formation is one of a surface groove and a passageway within the membrane.

146. The method of claim 145, wherein the surface groove or passageway within the membrane has a diameter of less than 100μ.

147. The method of claim 144, wherein the membrane is prepared by curing an elastomeric polymer in a mold containing a form defining the engineered structural formation.

148. The method of claim 147, wherein the form defining the engineered structural formation is a silicon wafer comprising a patterned photoresist layer defining the engineered structural formation.

149. The method of claim 147, comprising pouring PDMS over a silicon wafer comprising a patterned photoresist layer defining the engineered structural formation and heat curing the PDMS.

150. The method of claim 147, wherein the engineered structural formation is a channel.

151. The method of claim 144, wherein a second elastomeric layer is attached to the membrane.

152. The method of claim 151, wherein the engineered structural formation is a groove and the second elastomeric layer is aligned over the groove to form a passageway.

153. A method of culturing cells, comprising:
(a) growing cells in a suitable cell growth medium in a cell growth apparatus comprising a cell growth chamber having an interior side and an exterior side and comprising a wall and a base defining an interior volume, the cell growth chamber comprising an elastomeric growth substrate comprising an elastomeric membrane of a first material that comprises a first portion having a first elasticity and a second portion having a second elasticity and
(b) flexing the substrate while the cells are growing.

154. The method of claim 153, further comprising adding an analyte to the cell culture and determining the effect of the analyte on the cells.

155. A method of culturing cells, comprising:
(a) growing cells in a suitable medium in a cell growth apparatus comprising a cell growth chamber having an interior side and an exterior side and comprising a wall and a base defining an interior volume, the cell growth chamber comprising an elastomeric growth substrate comprising an elastomeric membrane of a first material that is at least partially coated with an extracellular matrix-mimetic; and
(b) flexing the substrate while the cells are growing.

156. The method of claim 155, further comprising adding an analyte to the cell culture and determining the effect of the analyte on the cells.

157. A method of culturing cells, comprising:
(a) growing cells in a suitable cell growth medium in a cell growth apparatus comprising a cell growth chamber having an interior side and an exterior side and comprising a wall and a base defining an interior volume, the cell growth chamber comprising an elastomeric growth substrate comprising a first elastomeric membrane and a removable second elastomeric membrane having one or more protuberances contacting the first elastomeric membrane or one or more openings, the periphery of which contact the first elastomeric membrane;
(b) flexing the substrate while the cells are growing; and
(c) removing the second elastomeric membrane.
